(19) United States
(12) Patent Application Publication
(10) Pub. No.: US 2011/0143336 A1
(43) Pub. Date: Jun. 16, 2011

(54) MARKER FOR ESTIMATING THE DIAGNOSIS OF CERVICAL ADENOCARCINOMA OR FOR ESTIMATING THE PROGNOSIS OF CERVICAL CANCER

(76) Inventors: Takashi Imai, Chiba (JP); Mayumi Iwakawa, Chiba (JP); Shingo Kato, Chiba (JP); Tatsuya Ohno, Chiba (JP)

(21) Appl. No.: 12/996,908

(22) PCT Filed: Oct. 10, 2008
(86) PCT No.: PCT/JP2008/068920

§ 371 Date: Feb. 9, 2011

(51) Int. Cl. C12Q 1/70; C12Q 1/68; G01N 33/53

(52) U.S. Cl. 435/5; 435/6.1; 435/7.1

(57) ABSTRACT

To provide a novel biomarker for estimating the diagnosis of cervical adenocarcinoma or for estimating the prognosis of cervical cancer. An antibody against Villin 1 is employed as a biomarker.

### TECHNICAL FIELD

The present invention relates to the use of an antibody against Villin1 as a marker for the diagnosis of cervical adenocarcinoma or the prognosis of cervical cancer.

### BACKGROUND ART

Cervical cancer is a type of uterine cancer and the second common malignancy among women (NON-PATENT DOCUMENT 1). Cervical cancer is classified into squamous cell carcinoma of the cervix and cervical adenocarcinoma. Cervical adenocarcinoma is a type of cervical cancer with glandular differentiation, includes not only typical adenocarcinoma derived from glandular epithelia cells but also adenosquamous carcinoma, and can be derived from all types of epithelia cells (NON-PATENT DOCUMENT 1). Adenosquamous carcinoma of the cervix is a type of cervical adenocarcinoma consisting of glandular epithelia-derived cancer cells and squamous epithelia-derived cancer cells, and accounts for 5 to 10% of all cervical cancers (NON-PATENT DOCUMENT 2).

The number of patients with cervical cancer in 2000 over the world was 470,600, and the number of deaths by the cancer was 233,400 (NON-PATENT DOCUMENT 3). The overall incidence and mortality rates of cervical cancer have significantly declined over the past three decades. However, recently, these declines have stopped, and the number of patients with cervical adenocarcinoma has risen slightly (NON-PATENT DOCUMENT 4).

Diagnostic tests for cervical cancer include a cytodiagnosis test, blood tests for various tumor markers, a tissue biopsy, and an imaging test. Among them, a smear screening with Papanicolaou staining and a human papillomavirus test have been widely done (for example, see NON-PATENT DOCUMENTS 5 and 6 to 15).

A Papanicolaou smear screening test is quite effective for the diagnosis of squamous cell carcinoma of the cervix. However, as for cervical adenocarcinoma, the test results in false-negative; therefore, it can not be indicative of cervical adenocarcinoma (for example, see NON-PATENT DOCUMENT 5). For this reason, a smear screening test is inappropriate for the diagnosis of cervical adenocarcinoma.

Furthermore, cervical adenocarcinoma has a similar histological appearance to early endometrial adenocarcinoma, and many subtypes of cervical adenocarcinoma are associated with endometrial differentiation. Due to this similar histological appearance, it is difficult to distinguish between endometrial adenocarcinoma and cervical adenocarcinoma by conventional tissue biopsies when the original tissue of tumors is obscure (see NON-PATENT DOCUMENT 1).

As candidate immunohistochemical staining biomarkers for cancer including cervical adenocarcinoma, MIB1, bcl-2, p16^{INK4a}, CEA, ER, vimentin and p53 have been investigated (for example, see NON-PATENT DOCUMENTS 6 to 11).

As for p16^{INK4a}, there has been reported that not only topical cervical adenocarcinoma and invasive cervical adenocarcinoma but also squamous epithelium dysplasia and squamous cell carcinoma were stained, whereby it was doubtful that p16^{INK4a} had the specificity for cervical adenocarcinoma (see NON-PATENT DOCUMENT 8). In fact, according to the investigation result by the present inventors as mentioned below, though p16^{INK4a} had high specificity for squamous cell carcinoma of the cervix, its specificity for cervical adenocarcinoma was insufficient. Incidentally, p16^{INK4a} immunohistochemical staining has been used for indirect assay of HPV infection (NON-PATENT DOCUMENT 10).

SCC antigen, CYFRA21-1 and CA125 have been clinically used as a marker for monitoring the post-treatment course of general gynecological tumors in a serological test. However, these have not been used as pre-treatment diagnostic markers for cervical adenocarcinoma (NON-PATENT DOCUMENT 11).

Oncogenic human papillomavirus (HPV) infection has been considered a cause of cervical cancer, and there has been reported that cervical cancer were related to many types of HPV, including types 6, 11, 16, 18, 52 and 58 (NON-PATENT DOCUMENT 12, etc.). Further, there has been reported that squamous cell carcinoma of the cervix had a distinct difference in oncogenic HPV types from cervical adenocarcinoma, and that, in western countries, infection of type 16 or 18 was found in 92% of virus-induced cervical adenocarcinoma, and especially infection of type 18 HPV was found in most cases (NON-PATENT DOCUMENT 1). Recently, 24-plex PathogenMip (NON-PATENT DOCUMENT 13) capable of identifying HPV genotypes and HPV linear array assay (NON-PATENT DOCUMENT 14) capable of identifying many HPV types have been developed as high through-put assays which are useful in determining HPV types.

However, according to the investigation result by the present inventors as mentioned below, cervical adenocarcinoma had a lower correlation with HPV infection, as compared to squamous cell carcinoma of the cervix. Accordingly, a HPV infection test was not sufficient in the specificity for cervical adenocarcinoma.

In addition to the above-mentioned pre-treatment diagnosis, in cervical cancer, especially in cervical adenocarcinoma, there has been need for the prognosis of cancerous lesion (NON-PATENT DOCUMENT 1).

In this respect, several researches have reported that early cervical adenocarcinoma had a poorer recuperation than squamous cell carcinoma of the cervix (NON-PATENT DOCUMENT 16). Also, there has been reported that histological classification was correlated with survival rates of patients with cervical cancer and can be a method for the prognosis of patients with cervical cancer (NON-PATENT DOCUMENT 17). Further, there have been reported that HPV infection can be significantly indicative of cervical cancer severity (NON-PATENT DOCUMENT 18) and that the HPV-negative patients among patients with cervical cancer had a significantly shorter lifetime than the HPV-positive patients among them (NON-PATENT DOCUMENT 19). Furthermore, there has been reported that adenosquamous carcinoma of the cervix had a poorer recuperation than typical cervical adenocarcinoma (NON-PATENT DOCUMENTS 20 and 21). On the other hand, some reports have described that it was not evidenced that histological classification was useful for the prognosis of patients with cervical cancer (NON-PATENT DOCUMENT 4).

### REFERENCES

NON-PATENT DOCUMENT 1: Herzog T J, Monk B J. Reducing the burden of glandular carcinomas of the uterine cervix. Am J Obstet Gynecol. 2007 December; 197(6):566-71.
NON-PATENT DOCUMENT 2: Shingleton H M, Bell M C, Fremgen A, et al. Is there really a difference in survival of women with squamous cell carcinoma, adenocarcinoma, and adenosquamous cell carcinoma of the cervix? Cancer 1995; 76:1948-55.
NON-PATENT DOCUMENT 3: Thomas G Cervical cancer: treatment challenges in the developing world. Radiother Oncol. 2006 May; 79(2):139-41.
NON-PATENT DOCUMENT 4: dos Reis R, Frumovitz M, Milam M R, Capp E, Sun C C, Coleman R L, Ramirez P T. Adenosquamous carcinoma versus adenocarcinoma in early-stage cervical cancer patients undergoing radical hysterectomy: an outcomes analysis. Gynecol Oncol. 2007 December; 107(3):458-63.
NON-PATENT DOCUMENT 5: Pak S C, Martens M, Bekkers R, Crandon A J, Land R, Nicklin J L, Perrin L C, Obermair A. Pap smear screening history of women with squamous cell carcinoma and cervical adenocarcinoma. Aust N Z J Obstet Gynaecol. 2007 December; 47(6):504-7.
NON-PATENT DOCUMENT 6: Negri G, Egarter-Vigl E, Kasal A, Romano F, Haitel A, Mian C. p16INK4a is a useful marker for the diagnosis of cervical adenocarcinoma uteri and its precursors: an immunohistochemical study with immunocytochemical correlations. Am J Surg Pathol. 2003 February; 27(2):187-93.
NON-PATENT DOCUMENT 7: Schorge J O, Lea J S, Elias K J, Rajanbabu R, Coleman R L, Miller D S, Ashfaq R. P16 as a molecular biomarker of cervical adenocarcinoma. Am J Obstet Gynecol. 2004 March; 190(3):668-73.
NON-PATENT DOCUMENT 8: Liang J, Mittal K R, Wei J J, Yee H, Chiriboga L, Shukla P. Utility of p16INK4a, CEA, Ki67, P53 and ER/PR in the differential diagnosis of benign, premalignant, and malignant glandular lesions of the uterine cervix and their relationship with Silverberg scoring system for endocervical glandular lesions. Int J Gynecol Pathol. 2007 January; 26(1):71-5.
NON-PATENT DOCUMENT 9: McCluggage W C, Sumathi V P, McBride H A, Patterson A. A panel of immunohistochemical stains, including carcinoembryonic antigen, vimentin, and estrogen receptor, aids the distinction between primary endometrial and endocervical adenocarcinomas. Int J Gynecol Pathol. 2002 January; 21(1):11-5
NON-PATENT DOCUMENT 10: Reid-Nicholson M, Iyengar P, Hummer A J, Linkov I, Asher M, Soslow R A. Immunophenotypic diversity of endometrial adenocarcinomas: implications for differential diagnosis. Mod Pathol. 2006 August; 19(8):1091-100.
NON-PATENT DOCUMENT 11: Gadducci A, Tana R, Fanucchi A, Genazzani A R. Biochemical prognostic factors and risk of relapses in patients with cervical cancer. Gynecol Oncol. 2007 October; 107(1 Suppl 1):S23-6.
NON-PATENT DOCUMENT 12: Walboomers J M, Jacobs M V, Manos M M, Bosch F X, Kummer J A, Shah K V, Snijders P J, Peto J, Meijer C J, Munoz N. Human papillomavirus is a necessary cause of invasive cervical cancer worldwide. J. Pathol. 1999 September; 189(1):12-9.
NON-PATENT DOCUMENT 13: Akhras M S, Thiyagarajan S, Villablanca A C, Davis R W, Nyren P, Pourmand N. PathogenMip assay: a multiplex pathogen detection assay. PLoS ONE. 2007 Feb. 21; 2(2):e223.
NON-PATENT DOCUMENT 14: Woo Y L, Damay I, Stanley M, Crawford R, Sterling J. The use of HPV Linear Array Assay for multiple HPV typing on archival frozen tissue and DNA specimens. J Virol Methods. 2007 June; 142(1-2):226-30.
NON-PATENT DOCUMENT 15: Malinowski D P. Molecular diagnostic assays for cervical neoplasia: emerging markers for the detection of high-grade cervical disease. Biotechniques. 2005 April; Suppl:17-23.
NON-PATENT DOCUMENT 16: Smith H O, Tiffany M F, Qualls C R, Key C R. The rising incidence of adenocarcinoma relative to squamous cell carcinoma of the uterine cervix in the United States—a 24-year population-based study. Gynecol Oncol. 2000 August; 78(2):97-105.
NON-PATENT DOCUMENT 17: Takeda N, Sakuragi N, Takeda M, Okamoto K, Kuwabara M, Negishi H, Oikawa M, Yamamoto R, Yamada H, Fujimoto S. Multivariate analysis of histopathologic prognostic factors for invasive cervical cancer treated with radical hysterectomy and systematic retroperitoneal lymphadenectomy. Acta Obstet Gynecol Scand. 2002 December; 81(12):1144-51.
NON-PATENT DOCUMENT 18: Spandidos D A, Dokianakis D N, Kallergi Aggelakis E. Molecular basis of gynecological cancer. Ann N Y Acad Sci. 2000; 900:56-64.
NON-PATENT DOCUMENT 19: Westra W H, Taube J M, Poeta M L, Begum S, Sidransky D, Koch W M. Inverse relationship between human papillomavirus-16 infection and disruptive p53 gene mutations in squamous cell carcinoma of the head and neck. Clin Cancer Res. 2008 Jan. 15; 14(2):366-9.
NON-PATENT DOCUMENT 20: Lea J S, Coleman R L, Garner E O, Duska L R, Miller D S, Schorge J O. Adenosquamous histology predicts poor outcome in low-risk stage IB1 cervical adenocarcinoma. Gynecol Oncol 2003; 91:558-62.
NON-PATENT DOCUMENT 21: Look K Y, Brunetto V L, Clarke-Pearson D L, et al. An analysis of cell type in patients with surgically staged stage 1B carcinoma of the cervix: a Gynecologic Oncology Group study. Gynecol Oncol 1996; 63:304-11.
NON-PATENT DOCUMENT 22: Iwakawa M, Ohno T, Imadome K, Nakawatari M, Ishikawa K, Sakai M, Katoh S, Ishikawa H, Tsujii H, Imai T. The radiation-induced cell-death signaling pathway is activated by concurrent use of cisplatin in sequential biopsy specimens from patients with cervical cancer. Cancer Biol Ther. 2007 June; 6(6):905-11.
NON-PATENT DOCUMENT 23: Ohno T, Nakano T, Niibe Y, Tsujii H, Oka K. Bax protein expression correlates with radiation-induced apoptosis in radiation therapy for cervical carcinoma. Cancer. 1998 Jul. 1; 83(1):103-10.

### DISCLOSURE OF THE INVENTION

### Means for Solving the Problems

The present inventors focused on an antibody against Villin1 (hereinafter abbreviated to VIL1) in the above-mentioned state of the art at the time of filing the present application. As a result of earnest investigation and repeated examinations using biopsy samples of cervical cancer patients, the present inventor found that the antibody is an effective marker for the diagnosis of cervical adenocarcinoma or the prognosis of cervical cancer. Based on the finding the present invention has been completed.

Thus, according to one embodiment of the present invention, there is provided a method for the diagnosis of cervical adenocarcinoma or the prognosis of cervical adenocarcinoma comprising the step of contacting a sample obtained from a patient with an antibody against VIL1, or a method for the diagnosis of cervical adenocarcinoma or the prognosis of cervical adenocarcinoma comprising the steps of contacting a cell obtained from the uterine cervix of a patient with an antibody against VIL1 and determining the presence of cervical adenocarcinoma or the prognosis thereof based on the expression levels of VIL1 in the cells.

In the method of the present invention, preferably, samples are tissues obtained from the uterine cervix of a patient. The method of the present invention, as combined with another diagnostic method, can achieve more efficient diagnosis or prognosis of cervical adenocarcinoma. The combination is useful for, for example, patients negative in the cytodiagnosis of cervical cancer with Papanicolaou staining, patients negative for human papillomavirus infection or patients negative for p16^{INK4a}. Also, advantageously, the method of the present invention can be combined with a method of detecting a mutation in a p53 tumor-suppressor gene to make a diagnosis of cervical adenocarcinoma or prognosis of cervical adenocarcinoma.

According to another embodiment of the present invention, there is provided a composition for the diagnosis of cervical adenocarcinoma or the prognosis of cervical cancer, comprising an antibody against VIL1.

According to still another embodiment of the present invention, there is provided a kit for the diagnosis of cervical adenocarcinoma or the prognosis of cervical adenocarcinoma, comprising an antibody against VIL1. According to still another embodiment of the present invention, there is provided the use of an antibody against VIL1 in vitro as a marker for the diagnosis of cervical adenocarcinoma or the prognosis of cervical adenocarcinoma.

According to still another embodiment of the present invention, there is provided the use of an antibody against VIL1 for preparing a composition for the diagnosis of cervical adenocarcinoma or the prognosis of cervical adenocarcinoma.

### BRIEF DESCRIPTION OF THE DRAWINGS

FIGS. 1A to 1C are graphs of Kaplan-Meier survival curves for 82 patients with cervical cancer, indicating that the lower the disease-free survival rate is, the poorer the prognosis is. FIG. 1A shows survival curves of two groups of patients positive for VIL1 staining and those negative for VIL1 staining. FIG. 1B shows survival curves of two groups of patients who are positive for VIL1 staining, negative for HPV infection and negative for 16^{INK4a} staining, and of the other patients. FIG. 1C shows survival curves of two groups of patients who are positive for VIL1 staining, negative for HPV infection and negative for 16^{INK4a} staining and have a mutation in p53, and of the other patients.

FIG. 2 is a graph showing the relative copy numbers resulted from quantitative PCR of VIL1 gene in the genomic DNAs from 93 cervical cancer patients, and comparing two histopathological classification groups of SCC and AD. SCC indicates cases of squamous cell carcinoma, and AD indicates cases of adenocarcinoma and adenosquamous carcinoma.

FIGS. 3A to 3E shows the results of avidin-biotin-based immunohistochemical staining according to one embodiment of the present invention, wherein the portions stained with brown indicates the presence of VIL1. FIG. 3A shows a histology of the health human small intestine as a control. FIG. 3B shows a histology of the health human uterine body. FIG. 3C shows a histology of the health human uterine cervix. FIG. 3D shows a histology of cervical adenocarcinoma. FIG. 3E shows a histology of squamous cell carcinoma of the cervix.

### BEST MODE FOR CARRYING OUT THE INVENTION

As mentioned above, the present invention relates to the use of an antibody against VIL1 for the diagnosis of cervical adenocarcinoma or the prognosis of cervical cancer. The present invention is described below.

### 1. VIL1

VIL1 is a calcium-regulated, actin-binding protein and belongs to the villin/gelsolin family. VIL1 is a cytoskeletal protein constituting a brush border and being expressed specifically in absorptive cells of the small intestine and in epithelial cells of the proximal renal tubule. VIL1 consists of a large core domain located at the center thereof, N-terminal side, C-terminal side and a small headpiece.

Human VIL1 gene is located at 2q35, and the amino acid sequence of a human VIL1 and the nucleic acid sequence of a cDNA encoding the amino acid sequence can be obtained or inferred from NCBI Sequence Viewer v2.0.

VIL1 can be isolated from tumor tissues by conventional protein extraction methods such as affinity chromatography. Further, sequencing methods thereof are well known to those skilled in the art.

### 2. Antibody

A primary antibody used in the present invention may be any of polyclonal and monoclonal antibodies which can be bound to VIL1.

As to an antibody which can be used in the present invention, many commercial products are distributed as shown in the following tables, and thus the commercial products can be conveniently used.

TABLE 1

| | | Product | Antigen/name | Antigen | Detailed source | Immunized | | |
|---|---|---|---|---|---|---|---|---|
| Importer | Manufacturer | name | of substance | species | information | animal | Isotype | Purity |
| Cosmo | Abcam | Villin | VIL, Villin1 | Chicken | Full length native | Mouse | IgG1? | Ig fraction-Protein |
| Bio Co., | | antibody | | | protein (purified) | Mono | | G |
| Ltd. | | [1D2C3] | | | (Chicken). | | | |
| Cosmo | Abcam | Villin | VIL, Villin1 | Human | Full length protein | Mouse | IgG1 | Protein G purified |
| Bio Co., | | antibody | | | (Human). | Mono | | |
| Ltd. | | [CWWB1] | | | | | | |
| | Abcam | Villin | VIL, Villin1 | Human | Full length protein | Mouse | IgG1? | Protein G purified |
| | | antibody | | | (Human) | Mono | | |
| | | [SPM226], | | | | | | |
| | | prediluted | | | | | | |
| | Abcam | Villin | VIL, Villin1 | Rabbit | Synthetic peptide | Rabbit | IgG | Protein A purified |
| | | antibody | | | corresponding to C- | Poly | | |
| | | | | | terminal of human | | | |
| | | | | | villin. | | | |
| | Abcam | Villin | VIL, Villin1 | Rabbit | Synthetic peptide | Rabbit | IgG | Immunogen affinity |
| | | antibody, | | | corresponding to C- | Poly | | purified |
| | | prediluted | | | terminal of human | | | |
| | | | | | villin. | | | |
| | abD serotec | MOUSE | VILLIN | Chicken | | Mouse | IgG1 | Purified IgG |
| | | ANTI | | | | Mono | | prepared by affinity |
| | | CHICKEN | | | | | | chromatography on |
| | | VILLIN | | | | | | Protein A |
| Funakoshi | Abnova | VIL1 | VIL1 (—, 1 a.a. | Human | Mouse polyclonal | Mouse | | |
| Co., Ltd. | Corporation | MaxPab(R) | ~421 a.a) full- | | antibody raised | Poly | | |
| | | polyclonal | length human | | against a full-length | | | |
| | | antibody | protein. | | human VIL1 | | | |
| | | (B01) | | | protein | | | |
| Funakoshi | Abnova | VIL1 | VIL1 | Human | Mouse monoclonal | Mouse | IgG2b | |
| Co., Ltd. | Corporation | monoclonal | (NP_009058, 1 | | antibody raised | Mono | | |
| | | antibody | a.a. ~78 a.a) | | against a partial | | | |
| | | (M02), clone | partial | | recombinant VIL1. | | | |
| | | 3G6 | recombinant | | | | | |
| | | | protein with | | | | | |
| | | | GST tag. | | | | | |
| Funakoshi | Abnova | VIL1 | VIL1 | Human | Mouse polyclonal | Mouse | | |
| Co., Ltd. | Corporation | polyclonal | (NP_009058, 1 | | antibody raised | Poly | | |
| | | antibody | a.a. ~78 a.a) | | against a partial | | | |
| | | (A01) | partial | | recombinant VIL1. | | | |
| | | | recombinant | | | | | |
| | | | protein with | | | | | |
| | | | GST tag. | | | | | |
| | ABR-Affinity | Villin | Villin | Human | Full length protein | Mouse | IgG1 | Protein G purified |
| | Bioreagents | | | | (Human) | Mono | | |
| | ABR-Affinity | Villin | Synthetic | Human | | Mouse | | affinity purified IgG |
| | Bioreagents | | peptide | | | Mono | | |
| | | | corresponding | | | | | |
| | | | to human | | | | | |
| | | | Villin. | | | | | |
| | ABR- | Villin | Purified chicken | Chicken | | Mouse Mono | IgG1 | |
| | Affinity | | villin | | | | | |
| | Bioreagents | | | | | | | |
| | ABR- | Villin | Human villin | Human | | Rabbit Poly | IgG | epitope affinity |
| | Affinity | | protein | | | | | purified IgG |
| | Bioreagents | | | | | | | |
| | Acris | Monoclonal | Human Villin | Human | | Mouse Mono | IgG1 | Protein G |
| | Antibodies | Antibody to | Protein | | | | | chromatography |
| | GmbH | Human Villin | | | | | | |
| | Acris | Monoclonal | Purified chicken | Chicken | | Mouse Mono | IgG1 | Protein A affinity |
| | Antibodies | Antibody to | Villin | | | | | chromatography |
| | GmbH | Villin | | | | | | |
| | Ana Spec | Anti-Villin | human Villin | Human | | Mouse Mono | IgG1? | purified from |
| | | | protein | | | | | ascites fluid by |
| | | | | | | | | Protein G |
| Sigma- | Atlas | Anti-VIL1 | Villin-1 | Human | | Rabbit Poly | IgG | Affinity purified |
| Aldrich | Antibodies | antibody | recombinant | | | | | using the PrEST- |
| Co. | | produced in | protein epitope | | | | | antigen as affinity |
| | | rabbit Ab1 | signature tag | | | | | ligand. |
| | | | (PrEST) | | | | | |
| Sigma- | Atlas | Anti-VIL1 | Villin-1 | Human | | Rabbit Poly | IgG | Affinity purified |
| Aldrich | Antibodies | antibody | recombinant | | | | | using the PrEST- |
| Co. | | produced in | protein epitope | | | | | antigen as affinity |
| | | rabbit Ab2 | signature tag | | | | | ligand. |
| | | | (PrEST) | | | | | |
| | BD | BD | Cow Villin aa. | Cow | | Mouse Mono | IgG1 | purified from tissue |
| | Biosciences | Transduction | 1-827 | | | | | culture supernatant |
| | | Laboratories ™ | | | | | | or ascites by |
| | | Villin | | | | | | affinity |
| | | | | | | | | chromatography. |
| | BECKMAN | VILLIN | villin | Human | | Monoclonal | | |
| | COULTER | | | | | | | |
| | BIOCARE | Villin, | Villin | | | Mouse Mono | IgG1? | |
| | MEDICAL | Concentrated | | | | | | |
| | | and Prediluted | | | | | | |
| | | Monoclonal | | | | | | |
| | | Antibody | | | | | | |
| | CELL | Villin(CWWB1) | Villin | | | Mouse | IgG1 | |
| | MARQUE | | | | | | | |
| Cell | Cell | Villin-1 | villin-1 | Human | Polyclonal | Rabbit | IgG, HRP- | purified using |
| Signaling | Signaling | (R814) | | | antibodies are | Poly | linked | protein A and |
| Technology | Technology | Antibody | | | produced by | | Antibody | peptide affinitty |
| Japan, K.K. | | | | | immunizing rabbits | | | chromatography. |
| | | | | | with a synthetic | | | |
| | | | | | peptide (KLH- | | | |
| | | | | | coupled) | | | |
| | | | | | corresponding to | | | |
| | | | | | the carboxy | | | |
| | | | | | terminus of human | | | |
| | | | | | villin-1. | | | |
| Cell | Cell | Villin-1 | villin-1 | Human | Polyclonal | Rabbit | IgG, HRP- | purified using |
| Signaling | Signaling | Antibody | | | antibodies are | Poly | linked | protein A and |
| Technology | Technology | | | | produced by | | Antibody | peptide affinitty |
| Japan, K.K. | | | | | immunizing rabbits | | | chromatography. |
| | | | | | with a synthetic | | | |
| | | | | | peptide (KLH- | | | |
| | | | | | coupled) | | | |
| | | | | | corresponding to | | | |
| | | | | | human villin-1. | | | |
| Funakoshi | GeneTex | VIL1 [3G6] | VIL1 | Human | | Mouse | IgG2b | Protein A affinity |
| Co., Ltd. | | antibody | (NP_009058, 1 | | | Mono | | purified |
| | | | a.a. ~78 a.a) | | | | | |
| | | | partial | | | | | |
| | | | recombinant | | | | | |
| | | | protein | | | | | |
| | GeneTex | VIL1 | VIL1 | | | Mouse | | Unpurified |
| | | antibody | (NP_009058, 1 | | | Poly | | |
| | | | a.a. ~78 a.a) | | | | | |
| | | | partial | | | | | |
| | | | recombinant | | | | | |
| | | | protein with GST | | | | | |
| | | | tag. | | | | | |
| | GenWay | MOUSE | Purified chicken | Chicken | | Mouse | IgG1 | Purified IgG |
| | Biotech, | ANTI | villin | | | Mono | | prepared by affinity |
| | Inc. | CHICKEN | | | | | | chromatography on |
| | | VILLIN | | | | | | Protein A |
| | | Antibody, | | | | | | |
| | | Mouse IgG | | | | | | |
| | | Monoclonal | | | | | | |
| | | Antibody | | | | | | |
| Cosmo Bio | LIFESPAN | Villin (VIL1) | Native | | Native protein | Mouse | IgG1 | Protein A column |
| Co., Ltd., | BIOSCIENCES | Mouse | protein(Villin | | | Mono | | |
| Funakoshi | INC. | Monoclonal | (VIL1)) | | | | | |
| Co., Ltd. | | Antibody | | | | | | |
| Cosmo Bio | LIFESPAN | Villin (VIL1) | Human Villin | Human | | Mouse | IgG1 | Affinity Purified |
| Co., Ltd., | BIOSCIENCES | Mouse anti- | protein(VIL1) | | | Mono | | |
| Funakoshi | INC. | Human | | | | | | |
| Co., Ltd. | | Monoclonal | | | | | | |
| | | Antibody | | | | | | |
| Cosmo | LIFESPAN | Villin (VIL1) | Purified | Chicken | Purified protein | Mouse | IgG1? | Purified |
| Bio Co., | BIOSCIENCES | Mouse anti- | chicken | | | Mono | | |
| Ltd., | INC. | Chicken | villin(VIL1) | | | | | |
| Funakoshi | | Monoclonal | | | | | | |
| Co., Ltd. | | Antibody | | | | | | |
| Cosmo | LIFESPAN | Villin (VIL1) | Purified | Chicken | Purified protein | Mouse | IgG1 | Affinity Purified |
| Bio Co., | BIOSCIENCES | Mouse anti- | chicken | | | Mono | | |
| Ltd., | INC. | Chicken | villin.(VIL1) | | | | | |
| Funakoshi | | Monoclonal | | | | | | |
| Co., Ltd. | | Antibody | | | | | | |
| | MILLIPORE | Anti-Villin, | Villin purified | Chicken | | Mouse | IgG1 | |
| | | clone 12 | from chicken | | | Mono | | |
| | | | intestine.(VIL1) | | | | | |
| | MILLIPORE | Anti-Villin, | Purified | Chicken | | Mouse | IgG1 | |
| | | clone ID2C3 | chicken | | | Mono | | |
| | | | villin(VIL1) | | | | | |
| Funakoshi | Novus | VIL1 | VIL1 | Human | | Mouse | IgG2b? | IgG purified |
| Co., Ltd. | Biologicals | antibody, | (NP_009058, | | | Mono | | |
| | | Mouse | 1 a.a. ~78 a.a) | | | | | |
| | | Monoclonal | partial | | | | | |
| | | anti-VIL1 | recombinant | | | | | |
| | | (3G6) | protein with | | | | | |
| | | | GST tag. | | | | | |
| Funakoshi | Novus | VIL1 | VIL1 | Human | | Mouse | | |
| Co., Ltd. | Biologicals | antibody, | (NP_009058, | | | Poly | | |
| | | Mouse | 1 a.a. ~78 a.a) | | | | | |
| | | Polyclonal | partial | | | | | |
| | | anti-VIL1 | recombinant | | | | | |
| | | | protein with | | | | | |
| | | | GST tag. | | | | | |
| Funakoshi | Novus | VIL1 | VIL1 (—, 1 a.a. | Human | | Mouse | | |
| Co., Ltd. | Biologicals | antibody, | ~421 a.a) full- | | | Poly | | |
| | | Mouse | length human | | | | | |
| | | Polyclonal | protein. | | | | | |
| | | anti-VIL1- | | | | | | |
| | | villin 1, | | | | | | |
| | | MaxPab | | | | | | |
| | | Antibody | | | | | | |
| Funakoshi | RayBiotech, inc. | MOUSE | Villin | Birds | | Mouse | | |
| Co., Ltd. | | ANTI | | | | Mono | | |
| | | VILLIN, With | | | | | | |
| | | HRP- | | | | | | |
| | | conjugated | | | | | | |
| | | secondary | | | | | | |
| | | antibody | | | | | | |
| | Santa Cruz | Villin | VIl1 | Chicken | purified full length | Mouse | | |
| | Biotechnology | (1D2C3) | | | native Villin of | Mono | | |
| | | Antibody | | | chicken origin. | | | |
| | Santa Cruz | Villin (C-19) | villin | Human | C-terminus | Goat Poly | IgG | |
| | Biotechnology | Antibody | | | | | | |
| | Santa Cruz | Villin | VIL1 | Human | full length Villin of | Mouse | IgG1 | |
| | Biotechnology | (CWWB1) | | | human origin. | Mono | | |
| | | Antibody | | | | | | |
| | Santa Cruz | Villin | VIL1 | Chicken | purified full length | Mouse | IgG1 | |
| | Biotechnology | (BDID2C3) | | | native Villin of | Mono | | |
| | | Antibody | | | chicken origin. | | | |
| | Santa Cruz | Villin (H-60) | VIL1 | Human | raised against | | IgG | |
| | Biotechnology | Antibody | | | amino acids | | | |
| | | | | | 721-780 | | | |
| | | | | | mapping near | | | |
| | | | | | the C-terminus of | | | |
| | | | | | Villin of human | | | |
| | | | | | origin. | | | |
| | Santa Cruz | Villin (K-11) | VIL1 | Human | raised against a | Goat Poly | IgG | |
| | Biotechnology | Antibody | | | peptide mapping at | | | |
| | | | | | the C-terminus of | | | |
| | | | | | Villin of human | | | |
| | | | | | origin. | | | |
| | Santa Cruz | Villin | VIL1 | Human | raised against | Mouse | IgG1 | |
| | Biotechnology | (SPM226) | | | recombinant Villin | Mono | | |
| | | Antibody | | | of human origin. | | | |
| | Santa Cruz | Villin (V-20) | VIL1 | Human | raised against a | Goat Poly | IgG | |
| | Biotechnology | Antibody | | | peptide mapping | | | |
| | | | | | near the N-terminus | | | |
| | | | | | of Villin of human | | | |
| | | | | | origin. | | | |
| | SPRING | Anti Villin | Villin | Human | | Mouse | IgG1? | |
| | BIOSCIENCE | | | | | | | |
| | SPRING | Anti Villin | Villin | Human | | Rabbit | | |
| | BIOSCIENCE | | | | | | | |
| | Thermo | Villin Ab-1 | Human Villin | Human | | Mouse | IgG1 | |
| | SCIENTIFIC | (Clone | protein | | | Mono | | |
| | | CWWB1) | | | | | | |
| | USBiological | Villin 1 Pab | villin-1 | Human | Synthetic peptide | Rabbit | IgG | Purified by Protein |
| | | Rb xHu | | | (KLH-coupled) | Poly | | A and |
| | | | | | corresponding to | | | immunoaffinity |
| | | | | | the carboxy | | | chromatography. |
| | | | | | terminus of human | | | |
| | | | | | villin-1. | | | |
| | USBiological | Villin Mab | Human Villin | Human | | Mouse | IgG1 | Purified by Protein |
| | | Mo xHu | protein | | | Mono | | G affinity |
| | | | | | | | | chromatography. |
| | YLEM S.R.L | Anti Villin | Villin | | | Mouse | IgG1 | |
| | Abnova | VIL1 | VIL1(AAH1 | | Length with Tag: | | | |
| | Corporation | Recombinant | 7303, 1 a.a. −422 | | 653 aa with GST | | | |
| | | Protein (P01) | a.a.) full- | | Tag | | | |
| | | | length | | | | | |
| | | | recombinant | | | | | |
| | | | protein with | | | | | |
| | | | GST. | | | | | |
| | Abnova | VIL1 | VIL1 | | Length with Tag: | | | |
| | Corporation | Recombinant | (NP_009058, | | 311 aa with GST | | | |
| | | Protein (Q01) | 1 a.a. −78 a.a.) | | Tag | | | |
| | | | partial | | | | | |
| | | | recombinant | | | | | |
| | | | protein with | | | | | |
| | | | GST. | | | | | |
| | Novus | VIL1 Partial | VIL1 | | | | | |
| | Biologicals | Recombinant | (NP_009058, | | | | | |
| | | Protein, VIL1 | 1 a.a. −78 a.a.) | | | | | |
| | | Partial | partial | | | | | |
| | | Recombinant | recombinant | | | | | |
| | | Protein (Q01) | protein with | | | | | |
| | | | GST. | | | | | |
| | Novus | VIL1 | VIL1(AAH1 | | | | | |
| | Biologicals | Recombinant | 7303, 1 a.a. −422 | | | | | |
| | | Protein | a.a.) full- | | | | | |
| | | | length | | | | | |
| | | | recombinant | | | | | |
| | | | protein with | | | | | |
| | | | GST. | | | | | |

TABLE 2

| Importer | Manufacturer | Product name | Clone | Species reactivity | Label | Applications |
|---|---|---|---|---|---|---|
| Cosmo | Abcam | Villin | 1D2C3 | Human, Chicken | Unlabeled | IHC paraffin |
| Bio Co., | | antibody | | | | embedding section, |
| Ltd. | | [1D2C3] | | | | IHC frozen section |
| Cosmo | Abcam | Villin | CWWB1 | Human | Unlabeled | IHC (Paraffin) |
| Bio Co., | | antibody | | | | |
| Ltd. | | [CWWB1] | | | | |
| | Abcam | Villin | SPM226 | Human | | IHC (Paraffin) |
| | | antibody | | | | |
| | | [SPM226], | | | | |
| | | prediluted | | | | |
| | Abcam | Villin | | Human | | IHC (Paraffin) |
| | | antibody | | | | |
| | Abcam | Villin | | Human | | IHC (Paraffin) |
| | | antibody, | | | | |
| | | prediluted | | | | |
| | abD serotec | MOUSE | ID2C3 | Human, Chicken, | | Immunohistology - |
| | | ANTI | | Pig | | Frozen, Western |
| | | CHICKEN | | | | Blotting |
| | | VILLIN | | | | |
| Funakoshi | Abnova | VIL1 | | Human | Unlabeled | WB |
| Co., Ltd. | Corporation | MaxPab(R) | | | | |
| | | polyclonal | | | | |
| | | antibody | | | | |
| | | (B01) | | | | |
| Funakoshi | Abnova | VIL1 | 3G6 | Human | Unlabeled | ELISA |
| Co., Ltd. | Corporation | monoclonal | | | | |
| | | antibody | | | | |
| | | (M02), clone | | | | |
| | | 3G6 | | | | |
| Funakoshi | Abnova | VIL1 | | Human, Other | Unlabeled | ELISA, |
| Co., Ltd. | Corporation | polyclonal | | species not tested. | | WB (Recombinant |
| | | antibody | | | | protein) |
| | | (A01) | | | | |
| | ABR- | Villin | CWWB1 | Human | | IHC (Paraffin) |
| | Affinity | | | | | |
| | Bioreagents | | | | | |
| | ABR- | Villin | SPM226 | Human | | IHC (Paraffin), |
| | Affinity | | | | | Immunoprecipitation |
| | Bioreagents | | | | | |
| | ABR- | Villin | ID2C3 | Human, | | IHC (frozen), WB |
| | Affinity | | | Chicken, | | |
| | Bioreagents | | | Porcine | | |
| | ABR- | Villin | | Human | | IHC (Paraffin) |
| | Affinity | | | | | |
| | Bioreagents | | | | | |
| | Acris | Monoclonal | CWWB1 | Human | | IHC (Paraffin) |
| | Antibodies | Antibody to | | | | |
| | GmbH | Human Villin | | | | |
| | Acris | Monoclonal | ID2C3 | Human, | | IHC (frozen), WB |
| | Antibodies | Antibody to | | Chicken, | | |
| | GmbH | Villin | | Porcine | | |
| | Ana Spec | Anti-Villin | | Human | | IHC |
| Sigma- | Atlas | Anti-VIL1 | | Human, Other | | IHC (formalin- |
| Aldrich | Antibodies | antibody | | species not | | fixed, paraffin- |
| Co. | | produced in | | tested. | | embedded |
| | | rabbit Ab1 | | | | sections), |
| | | | | | | Immunoblotting, |
| | | | | | | Protein array |
| Sigma- | Atlas | Anti-VIL1 | | Human, Other | | IHC (formalin- |
| Aldrich | Antibodies | antibody | | species not | | fixed, paraffin- |
| Co. | | produced in | | tested. | | embedded |
| | | rabbit Ab2 | | | | sections), |
| | | | | | | Immunoblotting, |
| | | | | | | Protein array |
| | BD | BD | 12 | Human | | IHC, IF, WB |
| | Biosciences | Transduction | | | | |
| | | Laboratories ™ | | | | |
| | | Villin | | | | |
| | BECKMAN | VILLIN | ID2C3 | Human | | Flow Cytometry |
| | COULTER | | | | | |
| | BIOCARE | Villin, | 1D2C3 | Human | | IHC (Paraffin) |
| | MEDICAL | Concentrated | | | | |
| | | and Prediluted | | | | |
| | | Monoclonal | | | | |
| | | Antibody | | | | |
| | CELL | Villin(CWWB1) | CWWB1 | Human | | IHC paraffin |
| | MARQUE | | | | | embedding |
| | | | | | | section, IHC |
| | | | | | | frozen section |
| Cell | Cell Signaling | Villin-1 | | Human, | | WB, |
| Signaling | Technology | (R814) | | Mouse, Rat | | Immunofluorescence |
| Technology | | Antibody | | | | (IF-IC) |
| Japan, K.K. | | | | | | |
| Cell | Cell Signaling | Villin-1 | | Human | | WB |
| Signaling | Technology | Antibody | | | | |
| Technology | | | | | | |
| Japan, K.K. | | | | | | |
| Funakoshi | GeneTex | VIL1 [3G6] | 3G6 | Human, | | ELISA |
| Co., Ltd. | | antibody | | Other | | |
| | | | | species not | | |
| | | | | tested. | | |
| | GeneTex | VIL1 | | Human, | | ELISA, WB |
| | | antibody | | Other | | |
| | | | | species not | | |
| | | | | tested. | | |
| | GenWay Biotech, | MOUSE | ID2C3 | Human, | | IHC (frozen), WB |
| | Inc. | ANTI | | Birds, Pig, | | |
| | | CHICKEN | | ChickenN.B. | | |
| | | VILLIN | | | | |
| | | Antibody, | | | | |
| | | Mouse IgG | | | | |
| | | Monoclonal | | | | |
| | | Antibody | | | | |
| Cosmo Bio | LIFESPAN | Villin (VIL1) | | Human, | | IHC, WB, Flow |
| Co., Ltd., | BIOSCIENCES | Mouse | | Chicken, Pig | | Cytometry |
| Funakoshi | INC. | Monoclonal | | | | |
| Co., Ltd. | | Antibody | | | | |
| Cosmo Bio | LIFESPAN | Villin (VIL1) | | Human | | IHC, IHC paraffin |
| Co., Ltd., | BIOSCIENCES | Mouse anti- | | | | embedding section |
| Funakoshi | INC. | Human | | | | |
| Co., Ltd. | | Monoclonal | | | | |
| | | Antibody | | | | |
| Cosmo Bio | LIFESPAN | Villin (VIL1) | | Human, | | IHC |
| Co., Ltd., | BIOSCIENCES | Mouse anti- | | Chicken, | | |
| Funakoshi | INC. | Chicken | | Porcine | | |
| Co., Ltd. | | Monoclonal | | | | |
| | | Antibody | | | | |
| Cosmo Bio | LIFESPAN | Villin (VIL1) | | Human, | | IHC, WB, IHC frozen |
| Co., Ltd., | BIOSCIENCES | Mouse anti- | | Chicken, | | section |
| Funakoshi | INC. | Chicken | | Porcine | | |
| Co., Ltd. | | Monoclonal | | | | |
| | | Antibody | | | | |
| | MILLIPORE | Anti-Villin, | 12 | Human, | | WB, |
| | | clone 12 | | Chicken | | Immunocytochemistry |
| | MILLIPORE | Anti-Villin, | ID2C3 | Human, | | IHC |
| | | clone ID2C3 | | Chicken, Pig | | |
| Funakoshi | Novus | VIL1 | 3G6 | Human, | Unconjugated | ELISA (Antibody |
| Co., Ltd. | Biologicals | antibody, | | Other | | reactive against |
| | | Mouse | | species not | | recombinant |
| | | Monoclonal | | tested. | | protein) |
| | | anti-VIL1 | | | | |
| | | (3G6) | | | | |
| Funakoshi | Novus | VIL1 | | Human, | Unconjugated | ELISA, WB (The |
| Co., Ltd. | Biologicals | antibody, | | Other | | quality control of |
| | | Mouse | | species not | | this antibody is |
| | | Polyclonal | | tested. | | limited to |
| | | anti-VIL1 | | | | Western blot on |
| | | | | | | the immunizing |
| | | | | | | protein.) |
| Funakoshi | Novus | VIL1 | | Human | Unconjugated | ELISA, |
| Co., Ltd. | Biologicals | antibody, | | | | WB (Antibody |
| | | Mouse | | | | Reactive Against |
| | | Polyclonal | | | | Tissue Lysate and |
| | | anti-VIL1- | | | | Transfected |
| | | villin 1, | | | | Lysate.) |
| | | MaxPab | | | | |
| | | Antibody | | | | |
| Funakoshi | RayBiotech, inc. | MOUSE | | | Unlabeled | |
| Co., Ltd. | | ANTI | | | | |
| | | VILLIN, | | | | |
| | | With HRP- | | | | |
| | | conjugated | | | | |
| | | secondary | | | | |
| | | antibody | | | | |
| | Santa Cruz | Villin | 1D2C3 | Human, | | WB, IP, IF, |
| | Biotechnology | (1D2C3) | | Chicken | | IHC (Paraffin), |
| | | Antibody | | | | ELISA |
| | Santa Cruz | Villin (C-19) | C-19 | Human, | | WB, IP, IF, |
| | Biotechnology | Antibody | | Mouse, | | IHC (Paraffin), |
| | | | | Rat, Cow | | ELISA |
| | Santa Cruz | Villin | CWWB1 | Human | | WB, IP, IF, |
| | Biotechnology | (CWWB1) | | | | IHC (Paraffin), |
| | | Antibody | | | | ELISA |
| | Santa Cruz | Villin | BDID2C3 | Human, | | WB, IP, IF, |
| | Biotechnology | (BDID2C3) | | Chicken, | | IHC (Paraffin), |
| | | Antibody | | Mouse | | ELISA |
| | Santa Cruz | Villin (H-60) | H-60 | Human, | | WB, IP, IF, solid |
| | Biotechnology | Antibody | | Mouse, Rat | | phase ELISA |
| | Santa Cruz | Villin (K-11) | K-11 | Human, | | WB, IF, solid |
| | Biotechnology | Antibody | | Mouse, Rat | | phase ELISA |
| | Santa Cruz | Villin | SPM226 | Human | | IF, IHC (Paraffin) |
| | Biotechnology | (SPM226) | | | | |
| | | Antibody | | | | |
| | Santa Cruz | Villin (V-20) | V-20 | Human, | | WB, IF, solid phase |
| | Biotechnology | Antibody | | Mouse, Rat | | ELISA |
| | SPRING | Anti Villin | SPM226 | Human | | IHC (Paraffin) |
| | BIOSCIENCE | | | | | |
| | SPRING | Anti Villin | | Human | | IHC (Paraffin) |
| | BIOSCIENCE | | | | | |
| | Thermo | Villin Ab-1 | CWWB1 | Human | | IHC (formalin-fixed, |
| | SCIENTIFIC | (Clone | | | | paraffin-embedded |
| | | CWWB1) | | | | sections) |
| | USBiological | Villin 1 Pab | | Human | | IHC, IF, WB |
| | | Rb xHu | | | | |
| | USBiological | Villin Mab | 3F392 | Human | | IHC |
| | | Mo xHu | | | | |
| | YLEM S.R.L | Anti Villin | CWWB1 | Human | | IHC (Paraffin) |
| | Abnova | VIL1 | | | | ELISA, |
| | Corporation | Recombinant | | | | WB(Recombinant |
| | | Protein (P01) | | | | protein), Antibody |
| | | | | | | Production, Array |
| | Abnova | VIL1 | | | | ELISA, |
| | Corporation | Recombinant | | | | WB (Recombinant |
| | | Protein (Q01) | | | | protein), Antibody |
| | | | | | | Production, Array |
| | Novus | VIL1 Partial | | | | ELISA, WB. Other |
| | Biologicals | Recombinant | | | | unit sizes are |
| | | Protein, VIL1 | | | | available. |
| | | Partial | | | | |
| | | Recombinant | | | | |
| | | Protein (Q01) | | | | |
| | Novus | VIL1 | | | | ELISA, WB. Other |
| | Biologicals | Recombinant | | | | unit sizes are |
| | | Protein | | | | available. |

| | | | Storage | |
|---|---|---|---|---|
| Importer | Manufacturer | Product name | temperature | |
| Cosmo | Abcam | Villin | 4° C. | Villin is a very |
| Bio Co., | | antibody | | specific marker for |
| Ltd. | | [1D2C3] | | gastrointestinal |
| | | | | tumors and |
| | | | | adenocarcinomas |
| | | | | of the pancreas |
| | | | | Other subsets of |
| | | | | tumors stained |
| | | | | with Villin are |
| | | | | Merkel cell, lung |
| | | | | (with rootlets), |
| | | | | ovarian and |
| | | | | kidney. It does |
| | | | | not stain breast |
| | | | | cancer. |
| Cosmo | Abcam | Villin | 4° C. | |
| Bio Co., | | antibody | | |
| Ltd. | | [CWWB1] | | |
| | Abcam | Villin | 4° C. | |
| | | antibody | | |
| | | [SPM226], | | |
| | | prediluted | | |
| | Abcam | Villin | 4° C. | |
| | | antibody | | |
| | Abcam | Villin | 4° C. | |
| | | antibody, | | |
| | | prediluted | | |
| | abD serotec | MOUSE | | |
| | | ANTI | | |
| | | CHICKEN | | |
| | | VILLIN | | |
| Funakoshi | Abnova | VIL1 | −20° C. or | |
| Co., Ltd. | Corporation | MaxPab(R) | lower | |
| | | polyclonal | | |
| | | antibody | | |
| | | (B01) | | |
| Funakoshi | Abnova | VIL1 | −20° C. | |
| Co., Ltd. | Corporation | monoclonal | | |
| | | antibody | | |
| | | (M02), clone | | |
| | | 3G6 | | |
| Funakoshi | Abnova | VIL1 | −20° C. or | |
| Co., Ltd. | Corporation | polyclonal | lower | |
| | | antibody | | |
| | | (A01) | | |
| | ABR- | Villin | 4° C. | |
| | Affinity | | | |
| | Bioreagents | | | |
| | ABR- | Villin | 4° C. | |
| | Affinity | | | |
| | Bioreagents | | | |
| | ABR- | Villin | 4° C. | |
| | Affinity | | | |
| | Bioreagents | | | |
| | ABR- | Villin | 4° C. | |
| | Affinity | | | |
| | Bioreagents | | | |
| | Acris | Monoclonal | 2-8° C., −20° C. | |
| | Antibodies | Antibody to | | |
| | GmbH | Human Villin | | |
| | Acris | Monoclonal | 2-8° C., −20° C. | |
| | Antibodies | Antibody to | | |
| | GmbH | Villin | | |
| | Ana Spec | Anti-Villin | 2-8° C. | |
| Sigma- | Atlas | Anti-VIL1 | −20° C. | |
| Aldrich | Antibodies | antibody | | |
| Co. | | produced in | | |
| | | rabbit Ab1 | | |
| Sigma- | Atlas | Anti-VIL1 | −20° C. | |
| Aldrich | Antibodies | antibody | | |
| Co. | | produced in | | |
| | | rabbit Ab2 | | |
| | BD | BD | −20° C. | |
| | Biosciences | Transduction | | |
| | | Laboratories ™ | | |
| | | Villin | | |
| | BECKMAN | VILLIN | | |
| | COULTER | | | |
| | BIOCARE | Villin, | 2-8° C. | Villin can be very |
| | MEDICAL | Concentrated | | useful in |
| | | and Prediluted | | differentiating colon |
| | | Monoclonal | | adenocarcinoma from |
| | | Antibody | | breast carcinoma and |
| | | | | from lung |
| | | | | adenocarcinoma. |
| | CELL | Villin(CWWB1) | 4° C. | |
| | MARQUE | | | |
| Cell | Cell Signaling | Villin-1 | −20° C. | |
| Signaling | Technology | (R814) | | |
| Technology | | Antibody | | |
| Japan, K.K. | | | | |
| Cell | Cell Signaling | Villin-1 | −20° C. | |
| Signaling | Technology | Antibody | | |
| Technology | | | | |
| Japan, K.K. | | | | |
| Funakoshi | GeneTex | VIL1 [3G6] | −20° C. or | |
| Co., Ltd. | | antibody | lower | |
| | GeneTex | VIL1 | | |
| | | antibody | | |
| | GenWay Biotech, | MOUSE | 4° C. or −20° C. | |
| | Inc. | ANTI | | |
| | | CHICKEN | | |
| | | VILLIN | | |
| | | Antibody, | | |
| | | Mouse IgG | | |
| | | Monoclonal | | |
| | | Antibody | | |
| Cosmo Bio | LIFESPAN | Villin (VIL1) | 4° C. or −20° C. | |
| Co., Ltd., | BIOSCIENCES | Mouse | | |
| Funakoshi | INC. | Monoclonal | | |
| Co., Ltd. | | Antibody | | |
| Cosmo Bio | LIFESPAN | Villin (VIL1) | | |
| Co., Ltd., | BIOSCIENCES | Mouse anti- | | |
| Funakoshi | INC. | Human | | |
| Co., Ltd. | | Monoclonal | | |
| | | Antibody | | |
| Cosmo Bio | LIFESPAN | Villin (VIL1) | | |
| Co., Ltd., | BIOSCIENCES | Mouse anti- | | |
| Funakoshi | INC. | Chicken | | |
| Co., Ltd. | | Monoclonal | | |
| | | Antibody | | |
| Cosmo Bio | LIFESPAN | Villin (VIL1) | | |
| Co., Ltd., | BIOSCIENCES | Mouse anti- | | |
| Funakoshi | INC. | Chicken | | |
| Co., Ltd. | | Monoclonal | | |
| | | Antibody | | |
| | MILLIPORE | Anti-Villin, | | |
| | | clone 12 | | |
| | MILLIPORE | Anti-Villin, | | |
| | | clone ID2C3 | | |
| Funakoshi | Novus | VIL1 | −20° C. or | |
| Co., Ltd. | Biologicals | antibody, | −80° C. | |
| | | Mouse | | |
| | | Monoclonal | | |
| | | anti-VIL1 | | |
| | | (3G6) | | |
| Funakoshi | Novus | VIL1 | −20° C. or | |
| Co., Ltd. | Biologicals | antibody, | −80° C. | |
| | | Mouse | | |
| | | Polyclonal | | |
| | | anti-VIL1 | | |
| Funakoshi | Novus | VIL1 | −20° C. or | |
| Co., Ltd. | Biologicals | antibody, | −80° C. | |
| | | Mouse | | |
| | | Polyclonal | | |
| | | anti-VIL1- | | |
| | | villin 1, | | |
| | | MaxPab | | |
| | | Antibody | | |
| Funakoshi | RayBiotech, inc. | MOUSE | | |
| Co., Ltd. | | ANTI | | |
| | | VILLIN, | | |
| | | With HRP- | | |
| | | conjugated | | |
| | | secondary | | |
| | | antibody | | |
| | Santa Cruz | Villin | 4° C. | |
| | Biotechnology | (1D2C3) | | |
| | | Antibody | | |
| | Santa Cruz | Villin (C-19) | 4° C. | |
| | Biotechnology | Antibody | | |
| | Santa Cruz | Villin | 4° C. or | |
| | Biotechnology | (CWWB1) | freeze | |
| | | Antibody | | |
| | Santa Cruz | Villin | 4° C. | |
| | Biotechnology | (BDID2C3) | | |
| | | Antibody | | |
| | Santa Cruz | Villin (H-60) | 4° C. | |
| | Biotechnology | Antibody | | |
| | Santa Cruz | Villin (K-11) | 4° C. | |
| | Biotechnology | Antibody | | |
| | Santa Cruz | Villin | 4° C. or | |
| | Biotechnology | (SPM226) | freeze | |
| | | Antibody | | |
| | Santa Cruz | Villin (V-20) | 4° C. | |
| | Biotechnology | Antibody | | |
| | SPRING | Anti Villin | 4° C. | |
| | BIOSCIENCE | | | |
| | SPRING | Anti Villin | 4° C. | |
| | BIOSCIENCE | | | |
| | Thermo | Villin Ab-1 | 2-8° C. | |
| | SCIENTIFIC | (Clone | | |
| | | CWWB1) | | |
| | USBiological | Villin 1 Pab | 4° C. or −20° C. | |
| | | Rb xHu | | |
| | USBiological | Villin Mab | 4° C. or −20° C. | |
| | | Mo xHu | | |
| | YLEM S.R.L | Anti Villin | 4° C. | |
| | Abnova | VIL1 | −80° C. | |
| | Corporation | Recombinant | | |
| | | Protein (P01) | | |
| | Abnova | VIL1 | −80° C. | |
| | Corporation | Recombinant | | |
| | | Protein (Q01) | | |
| | Novus | VIL1 Partial | −20° C. or | |
| | Biologicals | Recombinant | −80° C. | |
| | | Protein, VIL1 | | |
| | | Partial | | |
| | | Recombinant | | |
| | | Protein (Q01) | | |
| | Novus | VIL1 | −80° C. | |
| | Biologicals | Recombinant | | |
| | | Protein | | |

Preparation of a polyclonal antibody and a monoclonal antibody can be carried out in accordance with an instruction manual attached with a product, the relevant website of its distributor, or the like.

The method of the present invention can be carried out, for example, by an indirect method. Upon carrying out the invention by the indirect method, usually, an antibody against VIL1 may be used as a primary antibody, and an antibody against the primary antibody may be used as a secondary antibody.

A sample may be, for example, washed for 10 minutes three times with a 0.1M phosphate buffer solution followed by removing endogenous peroxidase therein with a 0.5% hydrogen peroxide solution, after that, washed again for 10 minutes three times with a 0.1M phosphate buffer solution, and then incubated with the serum from the same animal species as a species from which a secondary antibody is derived, for example, with a 0.1M phosphate buffer solution containing 2 to 10% of the normal serum, for 30 to 60 minutes, in order to inhibit non-specific response which causes background staining. Subsequently, a primary antibody solution adjusted with diluent, for example, a 0.1M phosphate buffer solution at a proper concentration is added to the sample, and the mixture is, for example, incubated for 24 to 76 hours. At this stage, the binding of the antibody may be directly detected, for example, by binding it to a fluorescent substance. However, indirect methods can be widely applied and can achieve highly sensitive detection.

Then, in case of a indirect method, a secondary antibody is added to the sample to visualize VIL1. Methods for visualization include the use of a fluorescently labeled secondary antibody, an enzyme method of allowing a secondary antibody to bound to be an enzyme followed by addition of a chromogenic substrate, and an ABC method of detecting a avidin-biotin complex by a biotinylated secondary antibody. These methods for labeling are well known to those skilled in the art. For instance, an instruction manual attached to a product, the relevant website of its distributor or the like would provide the details. In the ABC method, after incubation with a primary antibody, for instance, a sample may be washed for 10 minutes three times with a 0.1M phosphate buffer solution and then incubated with a biotinylated secondary antibody for 2 to 24 hours. The resultant sample containing avidin-biotin complexes may be washed again for 10 minutes three times with a 0.1M phosphate buffer solution and then incubated for 1 to 2 hours. The color thereof may be developed with diaminobenzidine (DAB) reaction solution. The DAB reaction solution may be prepared with, for example, a Tris buffer solution and be used after a hydrogen peroxide solution is added thereto. Ammonium nickel sulfate may be added.

### 3. Target Diseases

In the present invention, an antibody against VIL1 is used for determining the presence of cervical cancer or the prognosis thereof. Particularly, an antibody against VIL1 to be used in the present invention has high specificity for cervical adenocarcinoma and high correlations with the developments of cervical cancer in the future. Thus, it is particularly useful for these diagnosis and prognosis.

A diagnostic/prognostic method with an antibody against VIL1 according to the present invention in combination with another specific diagnostic or prognostic method enables a more efficient determination of the presence of cervical adenocarcinoma or a more efficient prognosis thereof.

For instance, though cytodiagnosis of cervical cancer with Papanicolaou straining is effective for the diagnosis of squamous cell carcinoma of the cervix, the test would give false-negative results to cervical adenocarcinoma (NON-PATENT DOCUMENT 1). Therefore, from the viewpoint of an efficient detection of cervical adenocarcinoma, it is advantageous to carry out the method of the present invention for patients diagnosed as negative by a cytodiagnosis of cervical cancer with Papanicolaou straining.

In Western countries, there has been reported that infection of types 16 and 18 of human papillomavirus enables a presumptive diagnosis of cervical adenocarcinoma. However, according to the investigation result by the present inventors, though Japanese patients with squamous cell carcinoma of the cervix were frequently associated with infection by them, Japanese patients with cervical adenocarcinoma showed the low frequency of infection by them. Thus, no correlation between them was found. Accordingly, human papillomavirus typing would not serve as an auxiliary diagnosis of cervical adenocarcinoma which Japanese patients suffer from. Therefore, it would be useful in efficiently detecting cervical adenocarcinoma to carry out the method of the present invention for the patients with no human papillomavirus infection.

Furthermore, in Western countries, the use of a papillomavirus vaccine in the near future is planned, and it is expected that this vaccine will drastically decrease cervical adenocarcinoma caused by human papillomavirus infection. Accordingly, cervical adenocarcinoma of which the cause is not human papillomavirus infection will become a main issue (NON-PATENT DOCUMENT 1).

For example, many types of human papillomavirus including types 6, 11, 16, 18, 52 and 58 have been considered a cause of cervical adenocarcinoma. The method according to the present invention can be carried out for patients diagnosed as negative for these types of human papillomavirus in order to address the above-mentioned issue.

Similarly, patients diagnosed as cervical adenocarcinoma by the method of the present invention may include patients diagnosed as negative by a p16^{INK4a} test. Therefore, in order to improve the detection rate of cervical adenocarcinoma, the method according to the present invention can be advantageously carried out for the patients diagnosed as negative by a p16^{INK4a} test.

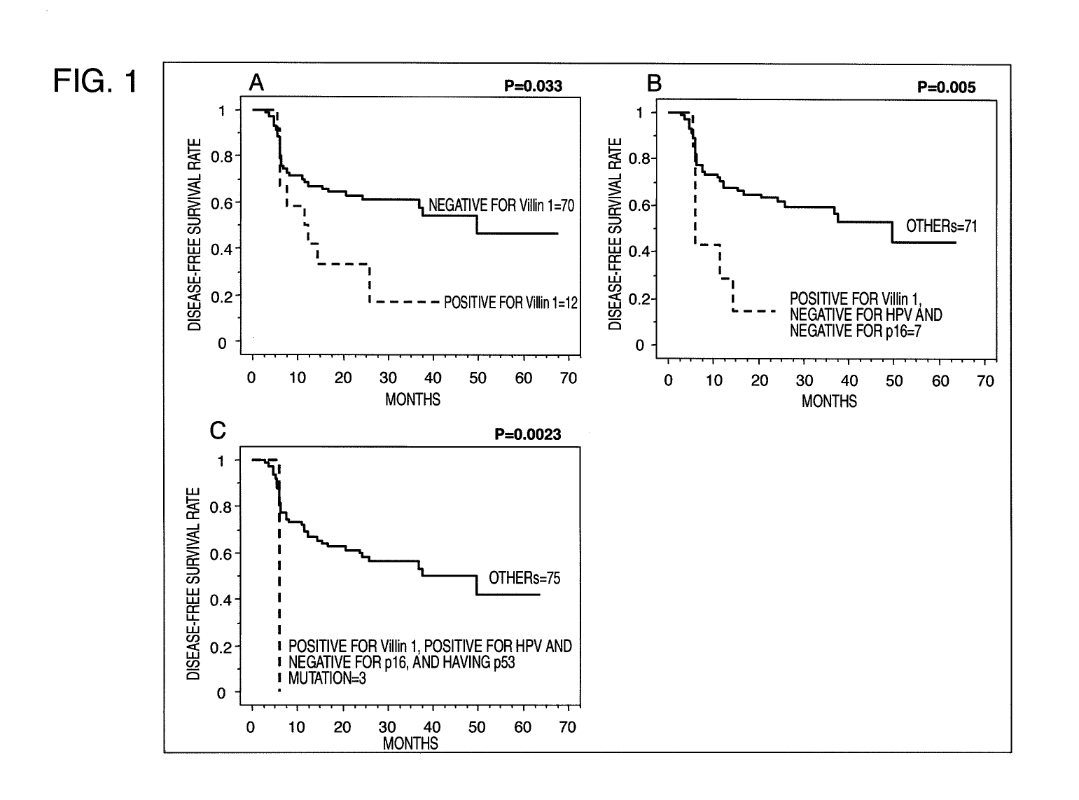

Furthermore, the present inventors have now found that A combination of a VIL1 test with HPV and/or p16^{INK4a} tests is useful for the prognosis of cervical cancer. Specifically, comparing disease-free survival rates of patients diagnosed as positive for VIL1 by the examination in which only VIL1 is used as a marker, with disease-free survival rates of patients diagnosed as positive for VIL1 and negative for HPV infection and/or p16^{INK4a} by the examination in which VIL1, and HPV and/or p16^{INK4a} are used as markers in combination; it was found that the latter patients had poorer disease-free survival rates as compared to the former ones (FIGS. 1A and 1B). Further, patients positive for VIL1 and negative for HPV infection and p16^{INK4a} and having a mutation in p53 had even poorer disease-free survival rates than patients positive for VIL1 and negative for HPV infection and p16^{INK4a} (FIG. 1C).

Accordingly, in order to make an accurate prognosis of cervical cancer, it is useful to combine a VIL1 test with at least one of HPV, p16^{INK4a} and p53 tests, preferably at least two of them, more preferably HPV and p16^{INK4a} tests, particularly preferably HPV, p16^{INK4a} and p53 tests.

### 4. Analysis and Diagnostic/Prognostic Procedure

The present invention relates to a method of making a diagnosis of cervical adenocarcinoma or a prognosis of cervical adenocarcinoma based on the level of expression of VIL1 in cells obtained from the uterine cervix of a patient. The method of the present invention includes the steps of contacting cells obtained from the uterine cervix of a patient with an antibody against VIL1 and measuring the amount of VIL1 bound to the antibody.

The amount of VIL1 bound to the antibody, i.e., the level of expression of VIL1 in cells obtained from the uterine cervix of a patient can be determined, for example, based on the presence or absence of or the level of color development of histochemical stain used as a label. Alternatively, it can be carried out by, for example, a ELISA method, automated flow cytometry, Western blot method or the like.

Further, a diagnosis of cervical adenocarcinoma or a prognosis of cervical adenocarcinoma according to the present invention may be made dependent on a used detection system. For instance, in a histochemical staining method, the diagnosis and prognosis can be carried out based on the following criteria.
0: Not stained
   1: Stained is the cell membrane of few tumor cells
   2: Stained is the cytoplasm and/or cell membrane in many tumor cells
   Synthetic judgement: 0 is negative and 1 and 2 are positive

### EXAMPLES

The present invention will be described below in more detail with reference to examples. However, it is to be noted that the following examples do not affect the technical scope of the present invention.

### 1. Samples

Among Japanese patients diagnosed as uterine cancer, which was developed at the uterine cervix, uterine body or vagina, and taking treatments at the National Institute of Radiological Sciences, Chiba, Japan; 122 patients who agreed with protocols issued by the institutional review board and gave informed consent to the use for research of their clinical records and biopsy tissues, participated in this study. The average age of the patients was 60. Among all the cases, 74 cases are squamous cell carcinoma of the cervix, 31 cases cervical adenocarcinoma and 5 cases uterine body adenocarcinoma.

Among 122 patients, 44 patients (including 31 cases of squamous cell carcinoma of the cervix, 11 cases of cervical adenocarcinoma and 2 cases of uterine body adenocarcinoma) received a 30.6 Gy radiotherapy to the whole pelvis and an additional 50.6 Gy radiotherapy to the pelvis with central shielding along with a ¹⁹²Ir high dose-rate intracavitary brachytherapy. Other 54 patients (including 33 cases of squamous cell carcinoma of the cervix, 12 cases of cervical adenocarcinoma, 1 case of uterine body adenocarcinoma and 8 cases of other types of uterine cancer) received the same treatments followed by 5 administrations of cisplatin at weekly intervals at the total amount of 40 mg/m². Other 24 patients (including 6 cases of squamous cell carcinoma of the cervix, 15 cases of cervical adenocarcinoma, 2 cases of uterine body adenocarcinoma and 1 case of other types of uterine cancer) received a 71.2 GyE carbon beam therapy.

Among all the patients, 110 patients were followed up for 2 years or more with regard to the recurrence and metastasis of the tumors.

It was found that 23 patients had distant metastasis (M1) and that 6 patients had large and invasive local tumors (T4). Therefore, they were excluded from the prognostic analysis. The excluded cases consisted of 19 cases of squamous cell carcinoma of the cervix, 5 cases of cervical adenocarcinoma, 0 case of uterine body adenocarcinoma and 3 cases of other types of uterine cancer. In one of these cases, the patient has distant metastasis (M1) and a large and invasive local tumor (T4).

All biopsy samples were collected from the uterine cervixes before the radiotherapy. Some biopsy samples were fixed with 10% formalin and then embedded in paraffin. Other biopsy samples were immersed in RNAlater, and genomic DNA was extracted from them (see NON-PATENT DOCUMENT 22).

### 2. Statistical Analysis

Fisher's exact test was used to analyze correlations during the respective tested parameters of VIL1 immunohistochemical detection, HPV infection, pathological classification, p53 mutation status and p16^{INK4a} immunohistochemical detection. P<0.05 was regarded as the “presence” of a significant difference. In a prognostic test, recurrence, metastasis and death in the two-year follow-up investigation were selected as evaluation items, and survival curves were plotted over time regarding the disease-free survival associated with no recurrence or metastasis according to the Kaplan-Meier method, the log-rank test being used to calculate significant differences.

### 3. Quantitative PCR Method

### 3-1. Procedure

PCR was carried out regarding tumor patients' DNAs and a reference DNA using a VIL1 gene as a primer. The genomic DNAs of 93 cervical cancer patients were extracted and purified from the biopsy samples in RNAlater with Genomic-tip (100/G) (QIAGEN). Commercially available, Human genomic DNA from females (Promega Corporation) was used as the reference DNA. As PCR reaction solutions, 30 ng of genomic DNA solutions were used. A primer was designed with ProbeFinder Software (Roche Diagnostics, Basel, Switzerland), and an appropriate hybridization probe was selected from Universal Probe Library (Roche). The nucleotide sequences of the used primers are shown in the following table.

TABLE 2
Primer sequences

| Name | | Primer | | Size of |
|---|---|---|---|---|
| of | | sequences | | product |
| gene | Direction * | (5′ > 3′) | Probe†† | (bp) |
| VIL1 | F | ggtgaagca | #72 | 78 |
| | | gggacacga | | |
| | R | ttggtgttact | | |
| | | ccacttgaagg | | |

Abbreviation:
F: Forward
R: Reverse
*: Direction of primer sequences
†Probe ID number of Universal Probe Library

LightCycler480 Probes Master (Roche) was used as a PCR reagent, and LightCycler480 (Roche) was used as a quantitative PCR device. The PCR reaction conditions were the following: denaturation of a DNA sample to a single-stranded form at 95° C. for 10 minutes followed by repeating 40 to 45 times a temperature cycle of 95° C. for 20 seconds, 55° C. or 60° C. for 30 seconds and 72° C. for 30 seconds. As a result of the temperature cycles, the target genes were amplified.

### 3-2. Result

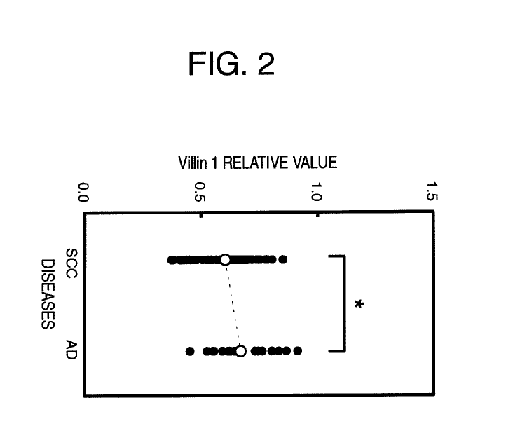

The copy number of VIL1 in a genomic DNA was divided by the copy number of VIL1 in the reference DNA to obtain relative value. In the relative value analysis of VIL1 copy number, a significant difference in copy numbers was found between squamous cell carcinoma and adenocarcinoma cases (P=0.049) (FIG. 2).

### 4. Immunohistochemical Staining

### 4-1. Staining Procedure and Criteria

Regarding VIL1 and p16^{INK4a}, streptavidin-biotin immunoperoxidase staining was carried out with an automatic staining machine, Ventana Discovery System (Ventana Medical Systems, Inc., Tucson, Ariz.) (see NON-PATENT DOCUMENT 23). Anti-VIL mouse monoclonal antibody (Cell Marque Corporation, CA) and anti-human p16^{INK4a} mouse monoclonal antibody (Thermo Fisher Scientific Inc., IPR, UK), which are primary antibodies, were diluted 100-fold with antibody dilution buffer (Ventana) to be used. Discovery Universal Secondary Antibody (Ventana) was used as a secondary antibody regarding both VIL1 and p16^{INK4a}. As a negative control, only a diluent, not containing primary antibodies, was used.

The respective evaluations of immunohistochemical staining were carried out in view of each of the staining patterns of VIL1 and p16^{INK4a} in accordance with the following criteria.

### 1) VIL1

0: Not stained
1: Stained is the cell membrane of few tumor cells
2: Stained is the cytoplasm and/or cell membrane in many tumor cells
Synthetic judgement: 0 was regarded as negative and 1 and 2 were regarded as positive
2) p16^{INK4a}
0: less than 1% of cells were stained at cell nucleus and cytoplasm
1: 1 to 10% of cells were stained at cell nucleus and cytoplasm; weak and diffuse staining
2: 10 to 30% of cells were strongly stained
3: more than 30% of cells were stained, and they were found in many areas in a specimen and strongly stained.
Synthetic judgement: 0, 1 and 2 were regarded as negative and 3 was regarded as positive

### 4-2. Result

### 1) VIL1

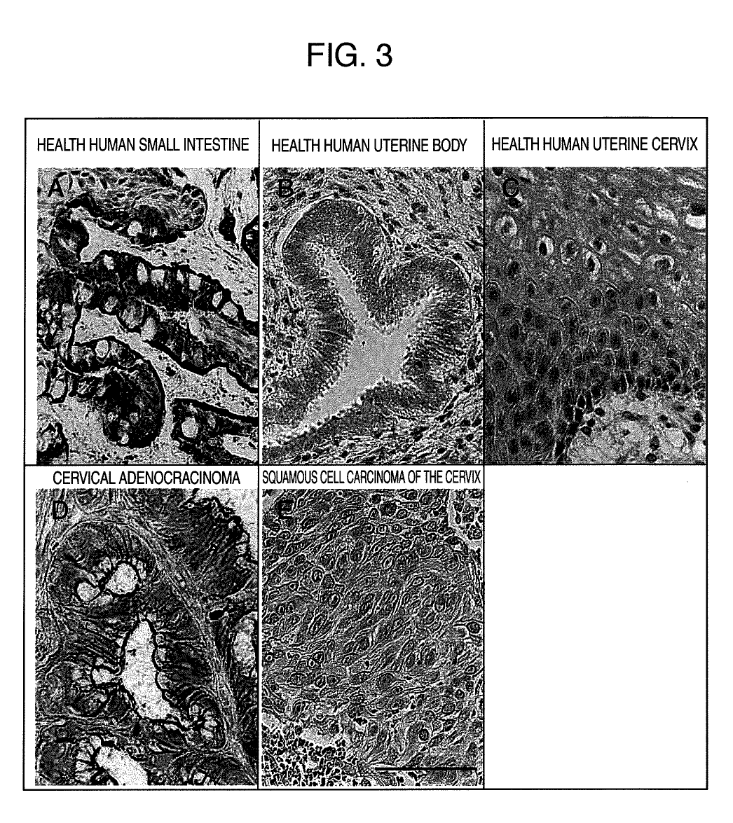

In the above-mentioned immunohistochemical staining, VIL1 was detected in the normal small intestinal epithelia (FIG. 3A) but was not found in the normal uterine body or uterine cervix (FIGS. 3B and 3C). Immunohistochemical staining was carried out on the formalin-fixed paraffin-embedded specimens of 122 cases. Some cases (13/31) of cervical adenocarcinoma were positive for VIL1 staining (FIG. 3D) while all cases (81/81) of squamous cell carcinoma were negative for VIL1 staining (FIG. 3E). There was a significant difference in VIL1 immunostaining positivity between squamous cell carcinoma of the cervix and cervical adenocarcinoma (P<0.0001). All VILI-positive tumors were cervical adenocarcinoma. VIL1 had the detection sensitivity of 41% and the selectivity of 100% as a diagnostic marker for cervical adenocarcinoma.

2) p16^{INK4a}

Regarding p16^{INK4a} staining, 114 cases were examined, and 97 cases (85%) among them were positive. Most cases (95%) of squamous cell carcinoma of the cervix were positive for p16^{INK4a}. In contrast, 52% of the cervical adenocarcinoma cases were negative for p16^{INK4a}.

### 5. Human Papillomavirus (HPV) Genotyping

### 5-1. Detection of HPV Infection by HPV Linear Array Assay

The target DNAs of HPV genes were amplified by PCR with biotinylated PGMY oligonucleotide probes in Linear Array HPV Genotyping Test (LA HPV GT, Roche). The amplified biotinylated products were detected by color development using Linear Array Detection Kit (LA DK, Roche). The identification by the color development was carried out by the steps of subjecting the amplified biotinylated product to hybridization with probes specific to 37 types of HPV and immobilized on a membrane to immobilize the amplified biotinylated product on the membrane, then adding streptavidin-horseradish peroxidase thereto to allow reaction thereof with biotin, and subsequently adding 3,3′ 5,5′-tetramethylbenzidine for blue color development.

### 5-2. Results

Ninety five (78%) out of 122 women had HPV-positive tumors. HPV-16 was most frequently detected. Seventy two out of 79 patients with squamous cell carcinoma of the cervix were HPV-positive while nineteen out of 37 patients with cervical adenocarcinoma were HPV-positive. The HPV-positive patients with squamous cell carcinoma of the cervix were significantly more than the HPV-positive patients with cervical adenocarcinoma (P<0.0001). Nine (64%) out of 14 VIL1 immunostaining positive cases were HPV-negative, and a correlation between HPV infection negative events and VIL1 staining positive events was found (P=0.0002).

### 6. p53 Mutation

### 6-1. Detection of Mutations in p53 Tumor-Suppressor Gene by High-Resolution Melting Analysis

A screening for detecting mutations in exons 5 to 8 of p53 gene was carried out regarding the DNA samples of 122 patents by high-resolution melting analysis. Exons 5 to 8 of p53 gene were amplified by PCR. The patients' DNA samples alone and mixtures thereof with an equal amount of reference DNA were used as a template DNA. 20 ul of PCR reaction solutions contained: 1×LightCycler480 High Resolution Melting Master (Roche); 2.5 mM of exon 5 in a MgCl₂ solution, 2.5 mM of exon 8 in a MgCl₂ solution, 3.0 mM of exon 6 in a MgCl₂ solution and 3.0 mM of exon 7 in a MgCl₂ solution, respectively; 0.2 uM of forward primer (see Table 2 as to its sequence); 0.2 uM of reverse primer (see Table 2 as to its sequence); and 10 ng of template DNA. LightCycler 480 thermal cycler (Roche) was used as a PCR reaction device. The reaction conditions were as follows: denaturation at 95° C. for 5 minutes followed by repeating 45 times a temperature cycle of 3 steps consisting of 95° C. for 10 seconds, 60° C. or 63° C. for 15 seconds and 72° C. for 10 seconds. Subsequently, melting (dissociation) curve analysis was carried out between 65° C. and 95° C. with the temperature transition rate of 4.8° C./s. The melting curve data was processed by LightCycler 480 Gene Scanning Software (Roche).

### 6-2. Result

Fifteen out of 122 cases had a mutation in a p53 tumor-suppressor gene. The VIL immunostaining positive samples had high, specifically 35%, mutation rate of p53 (P=0.0147). All of the VIL immunostaining positive cases having a mutation in p53 were negative for HPV-infection. Thus, it is supposed that cervical adenocarcinoma would include subtypes having a particular oncogenic mechanism which are positive for VIL1 but negative for HPV infection and have a mutation in p53.

### 7. Evaluation of VIL1 as a Prognostic Marker

In 82 patients with cervical cancer (including 50 cases of squamous cell carcinoma of the cervix and 25 cases of cervical adenocarcinoma) who could be followed up for 2 years or more after treatment, VIL1 was evaluated as a prognostic marker by carry outing an immunohistochemical staining test for specimens collected from them before the radiotherapy and post-therapeutic observation for a maximum of about 5 years after. As a result, it was found that VIL1 was useful as a prognosis-related factor (P=0.0122). As can be seen from Kaplan-Meier survival curves shown in FIGS. 1A to 1C, VIL1-staining positive tumors had a poor 2-year disease-free survival rate (P=0.033) (FIG. 1A), which demonstrated that the prognosis of cervical cancer were related to VIL1. Further, VIL-staining positive tumors which were negative for HPV infection and p16^{INK4a} had a poorer 2-year disease-free survival rate (P=0.005) (FIG. 1B), and VIL-staining positive tumors which were negative for HPV infection and p16^{INK4a} and had a mutation in p53 had a still poorer 2-year disease-free survival rate (P=0.0023) (FIG. 1C).

The foregoing results prove usefulness of VIL1 as a factor of the prognosis of cervical cancer and also proves that a combination of VIL1 with at least one of the presence or absence of HPV infection, the presence or absence of expression of p16^{INK4a} and the presence or absence of a mutation in p53 improves the accuracy of prognosis of cervical cancer.

1. A composition for diagnosis of cervical adenocarcinoma or prognosis of cervical cancer, comprising an antibody against Villin1.

2. The composition according to claim 1, wherein the diagnosis or the prognosis targets a patient diagnosed as negative by a cytodiagnosis of carcinoma of the uterine cervix with Papanicolaou straining.

3. The composition according to claim 1, wherein the diagnosis or the prognosis targets a patient negative for human papillomavirus infection.

4. The composition according to claim 3, wherein the human papillomavirus is any one of types 6, 11, 16, 18, 52 and 58.

5. The composition according to claim 4, wherein the human papillomavirus is type 16.

6. The composition according to claim 1, wherein the diagnosis or prognosis targets a patient negative for p16^{INK4a}.

7. The composition according to claim 1, for diagnosis of cervical adenocarcinoma or prognosis of cervical cancer in combination with a method for detecting a mutation in a p53 tumor-suppressor gene.

8. The composition according to claim 1, for prognosis of cervical cancer in combination with a human papillomavirus infection test and a p16^{INK4a} test.

9. The composition according to claim 1, for prognosis of cervical cancer in combined with a human papillomavirus infection test, a p16^{INK4a} test and a p53 tumor-suppressor gene test.

10. The composition according to claim 1, wherein the antibody against Villin1 is a monoclonal antibody.

11. The composition according to claim 1, wherein the antibody comprises a detectable label.

12. A kit for diagnosis of cervical adenocarcinoma or prognosis of cervical cancer, comprising an antibody against Villin1.

13. A method for making a prognosis of cervical adenocarcinoma or cervical cancer, comprising:
contacting a cell collected from uterine cervix of a patient with an antibody against Villin1; and
detecting Villin1 to which the antibody is bound.

14. The method according to claim 13, wherein the cell is derived from a patient diagnosed as negative by a cytodiagnosis of cervical carcinoma with Papanicolaou straining.

15. The method according to claim 13, wherein the cell is derived from a patient negative for human papillomavirus infection.

16. The method according to claim 13, wherein the human papillomavirus is any one of types 6, 11, 16, 18, 52 and 58.

17. The method according to claim 16, wherein the human papillomavirus is type 16.

18. The method according to claim 13, wherein the cell is derived from a patient negative for p16^{INK4a}.

19. A method for diagnosis of cervical adenocarcinoma or prognosis of cervical cancer, comprising conducting the method according to claim 13 in combination with a method for detecting a mutation in a p53 tumor-suppressor gene.

20. A method for prognosis of cervical cancer, comprising conducting the method according to claim 13 in combination with a human papillomavirus test and a p16^{INK4a} test.

21. A method for prognosis of cervical cancer, comprising conducting the method according to claim 13 in combination with a human papillomavirus infection test, a p16^{INK4a} test and a p53 tumor-suppressor gene test.
